(19) United States
(12) Patent Application Publication
(10) Pub. No.: US 2026/0015646 A1
(43) Pub. Date: Jan. 15, 2026

(54) ENZYME ELECTRODE, METHOD FOR PRODUCING ENZYME ELECTRODE, BIOSENSOR, AND BIO BATTERY

(71) Applicant: Murata Manufacturing Co., Ltd., Nagaokakyo-shi (JP)

(72) Inventors: Daisho TSUBOKAWA, Nagaokakyo-shi (JP); Hideyuki KUMITA, Nagaokakyo-shi (JP)

(21) Appl. No.: 19/334,019

(22) Filed: Sep. 19, 2025

Related U.S. Application Data
(63) Continuation of application No. PCT/JP2024/005769, filed on Feb. 19, 2024.

(30) Foreign Application Priority Data
Apr. 7, 2023 (JP) 2023-062870

(51) Int. Cl. C12Q 1/00; C12N 11/00; C12Q 1/26

(52) U.S. Cl. CPC C12Q 1/001; C12N 11/00; C12Q 1/26; C12Y 106/03001; C12Y 106/99

(57) ABSTRACT

An enzyme electrode that includes: an electrode support; an oxidoreductase; a conjugate of a silane coupling agent and an electron mediator; and a sol-gel matrix, wherein the oxidoreductase and the conjugate are fixed to the electrode support by the sol-gel matrix, and the silane coupling agent has a silicon atom, a reactive functional group, and a hydrolyzable group, and a structure in which the silicon atom and the reactive functional group are linked by a linking group having 4 or more carbon atoms.

### CROSS REFERENCE TO RELATED APPLICATIONS

The present application is a continuation of International application No. PCT/JP2024/005769, filed Feb. 19, 2024, which claims priority to Japanese Patent Application No. 2023-062870, filed Apr. 7, 2023, the entire contents of each of which are incorporated herein by reference.

### TECHNICAL FIELD

The present disclosure relates to an enzyme electrode, a method for producing an enzyme electrode, a biosensor, and a bio battery.

### BACKGROUND ART

In recent years, with growing interest in energy problems, bio batteries using bio-related substances such as sugars and alcohols as fuels have attracted attention. The bio battery uses an enzyme as an electrocatalyst, and can generate power by combining an oxidation reaction of the biofuel at the anode and a reduction reaction of oxygen or the like at the cathode.

In addition, for example, a biosensor using an enzyme electrode is used to measure a substance to be detected in a biological sample in various fields such as a medical field and a clinical examination field, and an oxidation-reduction reaction occurs between the substance to be detected, an oxidoreductase included in the enzyme electrode, and the substance to be measured, and a current thus generated is detected, thereby allowing the measurement.

In order to efficiently perform electron transfer in an enzyme electrode such as a bio battery or a biosensor, a technique performed by a substance called an electron mediator is known. In addition, in order to further improve the electron transfer efficiency, a technique for immobilizing an enzyme and a small molecule such as an electron mediator or an indicator on an electrode has also been developed. A sol-gel method is known as one of methods for immobilizing enzymes and the like, and there is a method for comprehensively immobilizing molecules by encapsulating in a matrix of metal oxides formed in a liquid.

Regarding the method for immobilizing an enzyme, Patent Document 1 discloses a method for immobilizing an enzyme in which an enzyme is immobilized on a structural unit having an inner diameter of 1.2 times or more the diameter of the enzyme and having structural stability, and then a net-like structure of a gelling substance is formed in a cavity and/or an internal void of the structural unit by a sol-gel method to improve the stability of the immobilized enzyme.

Patent Documents 2 and 3 disclose a technique of co-encapsulating a small molecule such as an electron mediator or an indicator with an enzyme for the purpose of sensing.

In the technique of Patent Documents 1 to 3, leakage of an enzyme can be suppressed, but there is a problem that small molecules such as an electron mediator leak from a gel matrix.

In order to solve the above problems, Non-Patent Document 1 discloses a method for preventing leakage of an electron mediator by covalently bonding an electron mediator as a small molecule to a sol-gel matrix.

Non-Patent Document 2 discloses that a sol-gel matrix is made hydrophobic in order to improve the stability of an immobilized enzyme.
Patent Document 1: Japanese Patent Application Laid-Open No. 2001-178457
Patent Document 2: Japanese Patent Application Laid-Open No. 2005-529309
Patent Document 3: Japanese Patent Application Laid-Open No. 2006-512573
Non-Patent Document 1: Z Wang et al., Biosens. Bioelectron., 2012, vol. 32, p. 111-117
Non-Patent Document 2: D Weiser et al., Green Chem., 2017, Vol. 19, p. 3927-3937

### SUMMARY OF THE DISCLOSURE

As described above, various techniques for immobilizing an enzyme or the like have been conventionally disclosed, but the enzyme electrode using the conventional immobilization method is not sufficient in terms of the electrocatalytic function, and there is room for improvement.

The present disclosure has been made in view of the above current situation, and an object thereof is to provide an enzyme electrode having an excellent electrocatalytic function as compared with a conventional enzyme electrode.

The present inventors have extensively conducted studies for achieving the above-described problems, and resultantly found that in an enzyme electrode using an oxidoreductase and an electron mediator, a bonded body obtained by bonding an electron mediator to a silane coupling agent having a structure in which a silicon atom and a reactive functional group are linked by a linking group having 4 or more carbon atoms is fixed to an electrode support by a sol-gel matrix, thereby leading to an excellent electrocatalytic function, and the present inventors have arrived at the solution of the above-described problems satisfactorily, and have reached the present disclosure.

The present disclosure is an enzyme electrode including: an electrode support; an oxidoreductase; a conjugate of a silane coupling agent and an electron mediator; and a sol-gel matrix, wherein the oxidoreductase and the conjugate are fixed to the electrode support by the sol-gel matrix, and the silane coupling agent has a silicon atom, a reactive functional group, and a hydrolyzable group, and a structure in which the silicon atom and the reactive functional group are linked by a linking group having 4 or more carbon atoms.

The enzyme electrode of the present disclosure includes the above-described configuration and has an excellent electrocatalytic function, and thus can be suitably used for a bio battery, a biosensor, and the like.

### BRIEF EXPLANATION OF THE DRAWINGS

FIG. 1 is a schematic view of an embodiment of an enzyme electrode of the present disclosure.

FIG. 2 is a schematic view showing an interaction between a silane coupling agent and an oxidoreductase in an embodiment of the enzyme electrode of the present disclosure.

FIG. 3 is a view showing voltammograms in cyclic voltammetry (CV) measurement using the enzyme electrodes obtained in Example 1 and Comparative Example 1.

FIG. 4 is a view showing a voltammogram in cyclic voltammetry (CV) measurement using the enzyme electrode obtained in Example 2.

FIG. 5 is a view showing a voltammogram in cyclic voltammetry (CV) measurement using the enzyme electrode obtained in Example 3.

FIG. 6 is a view showing analysis results by SAXS of compositions applied to electrode surfaces of enzyme electrodes obtained in Examples 2 and 3.

### DESCRIPTION OF THE PREFERRED EMBODIMENTS

Hereinafter, an enzyme electrode, a method for producing an enzyme electrode, a biosensor, and a bio battery of the present disclosure will be described.

However, the present disclosure is not limited to a configuration below, and can be appropriately modified and applied within a range in which the spirit of the present disclosure is not changed. The present disclosure also includes a combination of two or more of individual preferable configurations of the present disclosure described below.

### [Enzyme Electrode]

The enzyme electrode of the present disclosure includes an electrode support, an oxidoreductase, a conjugate of a silane coupling agent and an electron mediator, and a sol-gel matrix, wherein the oxidoreductase and the conjugate are fixed to the electrode support by the sol-gel matrix, and the silane coupling agent has a silicon atom, a reactive functional group, and a hydrolyzable group, and a structure in which the silicon atom and the reactive functional group are linked by a linking group having 4 or more carbon atoms.

For example, Non-Patent Document 1 discloses a technique in which an electron mediator is connected to a sol-gel matrix by a covalent bond, and leakage of the electron mediator to the outside of the matrix is suppressed, but a movable range of the electron mediator is limited, leading to deterioration of reactivity. In contrast, in the present disclosure, the silane coupling agent has a long-chain linking group as described above, and thus the electron mediator has a wide movable range and easily moves although fixed by a sol-gel matrix, and as a result, it is possible to efficiently perform electron transfer between an enzyme and enzyme and/or an enzyme and electrode. As a result, the catalytic current is improved, and when such an electrode is used for a battery, a sensor, or the like, the output is improved.

The content ratio of the conjugate of the silane coupling agent and the electron mediator in the enzyme electrode is preferably 1,000 mol % to 10 million mol % with respect to 100 mol % of the oxidoreductase. The content is more preferably 10,000 mol % to 1,000, 000 mol %, and still more preferably 100,000 mol % to 1,000,000 mol %.

### <Conjugate of Silane Coupling Agent and Electron Mediator>

The conjugate of the silane coupling agent and the electron mediator is not particularly limited as long as the silane coupling agent and the electron mediator are bonded, but it is preferable that the reactive functional group in the silane coupling agent and the reactive functional group in the electron mediator are bonded by a covalent bond.

### (Silane Coupling Agent)

The silane coupling agent is not particularly limited as long as it has a silicon atom, a reactive functional group, and a hydrolyzable group, and has a structure in which the silicon atom and the reactive functional group are linked by a linking group having 4 or more carbon atoms.

The reactive functional group of the silane coupling agent is not particularly limited as long as it can be bonded to an electron mediator, and examples thereof include a reactive functional group that interacts with an intermolecular force, a hydrogen bond, a Coulomb force, or the like, and a reactive functional group that can be covalently bonded, but the reactive functional group that can be covalently bonded is preferable from the viewpoint of the strength of bonding. Specific examples of the reactive functional group that can be covalently bonded include functional groups or reactive groups such as an amino group, a sulfonic acid group, a sulfuric acid group, a phosphoric acid group, a sulfhydryl group, a carboxyl group, groups of salts thereof, an epoxy group, a thiol group, a hydroxy group, a polymerizable unsaturated group, an azide group, an azo group, a nitro group, a nitrile group, a cyano group, an allene group, an isonitrile group, a urea group, an aldehyde group, a ketone group, a halogen group, a NHS ester, an imide ester, maleimide, a pyridyldithiol, an allyl azide, a haloacetate, an isocyanate, a carbodiimide, an allyl azide, a diazirine, a hydrazide, soralen, a pyridine disulfide, and a vinyl sulfone. Among these, an amino group, an epoxy group, and the like are preferable.

The silane coupling agent may have one or two or more types of the reactive functional groups.

The silane coupling agent has a linking group having 4 or more carbon atoms that links the silicon atom and the reactive functional group.

The linking group is not particularly limited as long as it is an organic group having 4 or more carbon atoms, and is preferably a hydrocarbon group having 4 to 30 carbon atoms and optionally having a hetero atom.

The linking group has such a structure, causing the linking group to become hydrophobic, and to lead to the hydrophobic interaction with the hydrophobic portion of the enzyme, allowing stabilization of the enzyme. This makes the enzyme electrode of the present disclosure excellent in durability when used as a battery or a sensor.

In addition, when the linking group has a long-chain hydrophobic group having 4 or more carbon atoms, hydration water in the vicinity of the active site of the enzyme is removed, and thus the electron transfer reaction rate between the enzyme and the electron mediator is further improved.

The hydrocarbon group is not particularly limited, and is preferably a group obtained by removing one or two or more hydrogen atoms from a linear or branched alkyl group, an alkenyl group, an alkynyl group, an aryl group, or the like.

Examples of the linear or branched alkyl group include a methyl group, an ethyl group, a n-propyl group, a n-butyl group, a n-pentyl group (amyl group), a n-hexyl group, a n-heptyl group, a n-octyl group, a n-nonyl group, a n-decyl group, a n-undecyl group, a n-dodecyl group, a n-tridecyl group, a n-tetradecyl group, a n-pentadecyl group, a n-hexadecyl group, a n-heptadecyl group, a n-octadecyl group, a n-nonadecyl group, a n-eicosanyl group, an i-propyl group, a sec-butyl group, an i-butyl group, a t-butyl group, a 1-methylbutyl group, a 1-ethylpropyl group, a 2-methylbutyl group, an i-amyl group, a neopentyl group, a 1,2-dimethylpropyl group, a 1,1-dimethylpropyl group, a t-amyl group, a 1,3-dimethylbutyl group, a 3,3-dimethylbutyl group, a 2-ethylbutyl group, a 2-ethyl-2-methylpropyl group, a 1-methylheptyl group, a 2-ethylhexyl group, a 1,5-dimethylhexyl group, a t-octyl group, a branched nonyl group, a decyl group, an undecyl group, a dodecyl group, a tridecyl group, a tetradecyl group, a pentadecyl group, a hexadecyl group, a heptadecyl group, a stearyl group, and an icosyl group.

Examples of the alkenyl group include a vinyl group, an allyl group, a 1-butenyl group, a 2-butenyl group, a pentenyl group, a hexenyl group, a heptenyl group, an octenyl group, a nonenyl group, a decenyl group, a dodecenyl group, an octadecenyl group, and an icosenyl group.

Examples of the alkynyl group include an ethynyl group, a 1-propynyl group, a 2-propynyl group, a butynyl group, a pentynyl group, a hexynyl group, a heptynyl group, an octynyl group, a nonynyl group, a decynyl group, a dodecynyl group, an octadecynyl group, and an icosynyl group.

Examples of the aryl group include a phenyl group; a naphthyl group; and aralkyl groups such as a benzyl group, a methylphenyl group, an ethylphenyl group, a propylphenyl group, a butylphenyl group, a butylmethylphenyl group, a dimethylphenyl group, a diethylphenyl group, a dibutylphenyl group, a biphenyl group, a methylbiphenyl group, an ethylbiphenyl group, a methylnaphthyl group, an ethylnaphthyl group, a cinnamyl group (Ph-CH═CHCH₂— group), and a 1-benzocyclobutenyl group.

The number of carbon atoms in the hydrocarbon group is preferably 5 to 25, more preferably 6 to 20, still more preferably 7 to 18, and particularly preferably 8 to 15.

The hydrocarbon group may have a hetero atom such as a nitrogen atom, a sulfur atom, an oxygen atom, a phosphorus atom, or a halogen atom, and may have a substituent including a hetero atom such as a hydroxy group, an alkoxy group, a carboxyl group, an acyl group, a sulfonic acid group, an amino group, or a phosphoric acid group.

The silane coupling agent preferably has a positive or negative charge. This makes it possible to further stabilize the enzyme and maintain the enzyme activity for a longer time by electrostatic interaction with a positively- or negatively-charged site on the enzyme surface.

The silane coupling agent preferably has a functional group having a positive or negative charge, and the functional group having a positive or negative charge may be in any portion of the silane coupling agent. The silane coupling agent preferably has a positive or negative charge in the linking group and/or the reactive functional group. When the reactive functional group has a positive or negative charge, the reactive functional group may have a positive or negative charge in the structure after binding to the electron mediator.

When the reactive functional group in the silane coupling agent has a positive or negative charge and has a hydrocarbon group having 4 or more carbon atoms as the linking group, the enzyme can be further stabilized by electrostatic interaction and hydrophobic interaction.

Examples of the functional group having a positive charge include primary to tertiary amino groups and quaternary ammonium groups.

Examples of the functional group having a negative charge include an epoxy group, an ether group, a carboxyl group, a sulfonic acid group, a sulfuric acid group, a phosphoric acid group, a thiol group, and a halogen group.

The silane coupling agent preferably has a structure in which a hydrolyzable group such as an alkoxy group is bonded to a silicon atom, and more preferably has a structure represented by the following formula (1):

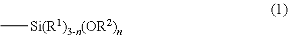

(In formula (1), R¹ and R² are the same or different and represent a hydrocarbon group having 1 to 5 carbon atoms. n represents an integer of 1 to 3).

n is an integer of 1 to 3, preferably 2 or 3, and more preferably 3.

The hydrocarbon group in R¹ and R² in the above formula (1) is not particularly limited, and examples thereof include an alkyl group, an alkenyl group, and an aryl group. Specific examples thereof include those described in the above linking group.

The number of carbon atoms of the hydrocarbon group in R¹ and R² is preferably 1 to 4, more preferably 1 to 3, and still more preferably 1 to 2.

The hydrocarbon group in R¹ and R² is preferably an alkyl group, more preferably a methyl group, an ethyl group, a n-propyl group, or a n-butyl group, and more preferably a methyl group or an ethyl group.

Examples of the silane coupling agent include: amino group-containing silane coupling agents such as N-2-(aminoethyl)-8-aminooctyltrimethoxysilane, N-2-(aminoethyl)-8-aminooctylmethyldimethoxysilane, N-2-(aminoethyl)-8-aminooctyldimethylmethoxysilane, N-2-(aminoethyl)-8-aminooctyltriethoxysilane, N-β-(aminoethyl)-γ-aminopropyltrimethoxysilane, N-β-(aminoethyl)-γ-aminopropylmethyldimethoxysilane, N-β-(aminoethyl)-γ-aminopropyltriethoxysilane, N-β-(aminoethyl)-γ-aminopropylmethyldiethoxysilane, N-β-(aminoethyl)-γ-aminopropyltriisopropoxysilane, 4-aminobutyltrimethoxysilane, 4-aminobutyltriethoxysilane, 5-aminopentyltrimethoxysilane, 5-aminopentyltriethoxysilane, 6-aminohexyltrimethoxysilane, 6-aminohexyltriethoxysilane, 7-aminoheptyltrimethoxysilane, 7-aminoheptyltriethoxysilane, 8-aminooctyltrimethoxysilane, and 8-aminooctyltriethoxysilane; and epoxy group-containing silane coupling agent such as 8-glycidoxyoctyltrimethoxysilane, 8-glycidoxyoctylmethyldimethoxysilane, 8-glycidoxyoctylmethyldiethoxysilane, 8-glycidoxyoctyltriethoxysilane, 7-glycidoxyheptyltrimethoxysilane, 7-glycidoxyheptylmethyldimethoxysilane, 7-glycidoxyheptylmethyldiethoxysilane, 7-glycidoxyheptyltriethoxysilane, 6-glycidoxyhexyltrimethoxysilane, 6-glycidoxyhexylmethyldimethoxysilane, 6-glycidoxyhexylmethyldiethoxysilane, 6-glycidoxyhexyltriethoxysilane, 5-glycidoxypentyltrimethoxysilane, 5-glycidoxypentylmethyldiethoxysilane, 5-glycidoxypentylmethyldiethoxysilane, 5-glycidoxypentyltriethoxysilane, 4-glycidoxybutyltrimethoxysilane, 4-glycidoxybutylmethyldimethoxysilane, 4-glycidoxybutylmethyldiethoxysilane, 4-glycidoxybutyltriethoxysilane, γ-glycidoxypropyl (ethyl)dimethoxysilane, β-3,4-epoxycyclohexylethyltrimethoxysilane, and β-3,4-epoxycyclohexylethyltriethoxysilane.

### (Electron Mediator)

In the enzyme electrode of the present disclosure, the electron mediator that binds to the silane coupling agent is not particularly limited as long as it exchanges electrons with the oxidoreductase included in the enzyme electrode of the present disclosure, and can bind to a reactive functional group in the silane coupling agent, and preferably has a functional group that forms a bond with a reactive functional group of a silane coupling agent such as an amino group, a carboxyl group, or an aldehyde group.

Examples of the electron mediator include coenzymes of oxidoreductase and an electron transfer agent other than coenzymes.

Specific examples of the coenzyme of the oxidoreductase include vitamin coenzymes such as nicotinamide adenine dinucleotide, nicotinamide adenine dinucleotide phosphate, flavin adenine dinucleotide, and flavin mononucleotide; and quinone coenzymes such as pyrroloquinoline quinone, topaquinone, tryptophan-tryptophylquinone, lysinyrosylquinone, and cysteinyl-tryptophan quinone.

In the present specification, an oxidized form of nicotinamide adenine dinucleotide is also referred to as NAD, a reduced form thereof is also referred to as NADH, an oxidized form of nicotinamide adenine dinucleotide phosphate is also referred to as NADP, and a reduced form thereof is also referred to as NADPH.

In addition, NAD(P)H means NADH or NADPH, and NAD(P) means NAD or NADP. In addition, NAD means NAD⁺, and NADP means NADP⁺.

Examples of the electron transfer agent other than the coenzyme include metal elements such as Os, Fe, Ru, Co, Cu, Ni, V, Mo, Cr, Mn, Pt, and W, or metal complexes having ions of these metals as central metals (ferrocene and alkali metals ferricyanide such as potassium ferricyanide, lithium ferricyanide, and sodium ferricyanide, or alkyl substitutes thereof (methyl substituted compound, ethyl substituted compound, propyl substituted compound, and the like), potassium octacyanotungstate, and the like); quinones such as quinone, benzoquinone, anthraquinone, naphthoquinone, and aminonaphthoquinone; heterocyclic compounds such as toluidine blue, methylene blue, viologen, methyl viologen, benzyl viologen, phenazine methosulfate, phenazine ethosulfate, bipyridine or derivatives thereof; and 2,6-dichlorophenolindophenol, methylene blue, and potassium β-naphthoquinone-4-sulfonate.

As the above metal complex, a metal complex having iron as a central metal is preferable, and ferrocenes such as aminoferrocene and ferrocenecarboxaldehyde are more preferable.

When the enzyme electrode of the present disclosure includes an oxidoreductase (A) of a substrate described later, it is preferable to use an electron mediator (a) that is a coenzyme of the oxidoreductase (A).

When the coenzyme of the oxidoreductase (A) of the substrate is NAD(P), that is, when the enzyme electrode of the present disclosure includes an NAD(P)-dependent oxidoreductase, it is preferable to use NAD(P) as the electron mediator (a).

When the enzyme electrode of the present disclosure further includes an enzyme (B) that receives electrons from an electron mediator (a) such as a coenzyme and transmits the received electrons to the electrode support, or transmits electrons received from the electrode support to the electron mediator (a), it is preferable to use an electron mediator (b) that mediates electron transmission between the enzyme (B) and the electrode support.

An embodiment in which the electron mediator includes NAD(P)H and/or NAD(P) as the electron mediator (a) and an electron mediator other than NAD(P)H and NAD(P) as the electron mediator (b) is one of preferable embodiments of the present disclosure.

The electron mediator (b) is not particularly limited, and is preferably a heterocyclic compound having a phenothiazine skeleton such as quinones, ferrocenes, or toluidine blue. Quinones such as aminonaphthoquinone are more preferable.

It is a preferable embodiment of the present disclosure that the enzyme electrode includes a conjugate of a silane coupling agent and an electron mediator (a) such as a coenzyme (hereinafter, also referred to as a conjugate (α)) and a conjugate of a silane coupling agent and an electron mediator (b) (hereinafter, also referred to as a conjugate (B)).

### <Oxidoreductase>

The oxidoreductase is not particularly limited as long as it exchanges electrons with an electron mediator, and the enzyme electrode of the present disclosure preferably includes an enzyme (A) (hereinafter, also referred to as oxidoreductase (A) or simply enzyme (A)) that oxidizes and/or reduces a substrate described later and exchanges electrons with an electron mediator. The enzyme (A) is more preferably an oxidase for a substrate. More preferable is NAD(P)-dependent oxidase (hereinafter, also referred to as an NAD(P)-dependent oxidase) that exchanges electrons with NAD(P)H or NAD(P).

Preferable examples of the oxidase include glycerol dehydrogenase, glucose dehydrogenase, a series of enzymes of an electron transfer system, an ATP synthase, and an enzyme involved in sugar metabolism (for example, hexokinase, glucose phosphate isomerase, phosphofructokinase, fructose diphosphate aldolase, triose phosphate isomerase, glyceraldehyde phosphate dehydrogenase, phosphoglycerate mutase, phosphopyruvate hydratase, pyruvate kinase, L-lactate dehydrogenase, D-lactate dehydrogenase, pyruvate dehydrogenase, citrate synthase, aconitase, isocitrate dehydrogenase, 2-oxoglutarate dehydrogenase, succinyl-CoA synthetase, succinate dehydrogenase, fumarase, and malonate dehydrogenase). These can be used singly or in combination of two or more.

Among these, glycerol dehydrogenase and glucose dehydrogenase are preferable.

The enzyme electrode of the present disclosure preferably further includes an enzyme (B) (hereinafter, also referred to as oxidoreductase (B) or simply enzyme (B)) that receives electrons from the electron mediator (a) and transfers the received electrons to the electrode support, or transfers the electrons received from the electrode support to the electron mediator (a).

The enzyme electrode of the present disclosure preferably includes diaphorase as the enzyme (B).

Diaphorase is an enzyme that catalyzes the oxidation-reduction reaction of the oxidation-reduction pair of NAD(P) and can exchange electrons with the electron mediator (b) or the electrode surface.

When the enzyme electrode of the present disclosure includes an NAD(P)-dependent oxidase and a diaphorase, NAD(P) receives an electron generated by oxidation of a substrate, NAD(P) becomes NAD(P)H, the NAD(P)H transfers an electron to the diaphorase, the diaphorase transfers an electron to the electron mediator (b), and the electron mediator (b) transfers an electron to the electrode support, thereby allowing the electron extracted from the substrate to be transferred to the electrode support.

When the oxidoreductase includes the oxidoreductase (A) and the oxidoreductase (B), the content ratio of the oxidoreductase (B) is preferably 10 mol % to 1,000 mol % with respect to 100 mol % of the oxidoreductase (A). The content ratio is more preferably 20 mol % to 500 mol %, and still more preferably 30 mol % to 100 mol %.

The enzyme electrode preferably includes the conjugate (α) and the conjugate (β), and the enzyme (A) and the enzyme (B).

In this case, the content ratio of the conjugate (α) is not particularly limited, and is preferably 1,000 mol % to 10,000,000 mol % with respect to 100 mol % of the enzyme (A). The content is more preferably 10,000 mol % to 1,000, 000 mol %, and still more preferably 100,000 mol % to 1,000,000 mol %.

In addition, the content ratio of the conjugate (β) is not particularly limited, and is preferably 1,000 mol % to 10,000,000 mol % with respect to 100 mol % of the enzyme (B). The content is more preferably 10,000 mol % to 1,000, 000 mol %, and still more preferably 100,000 mol % to 1,000,000 mol %.

A form in which the enzyme electrode includes the conjugate (α) and conjugate (β), and the NAD-dependent oxidase and diaphorase is also one of preferable embodiments of the present disclosure.

### <Sol-Gel Matrix>

The enzyme electrode of the present disclosure includes a sol-gel matrix.

The sol-gel matrix is not particularly limited as long as it can fix the oxidoreductase and the conjugate of the silane coupling agent and the electron mediator to the electrode support, and preferably includes a silane compound.

The silane compound preferably has a structure in which a hydrolyzable group such as an alkoxy group is bonded to a silicon atom, and more preferably is a silane compound having an alkoxy group, represented by the following formula (2):

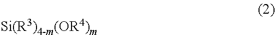

(In the formula (2), R³ and R⁴ are the same or different and represent a hydrocarbon group having 1 to 5 carbon atoms, and m represents an integer of 1 to 4).

The hydrocarbon group in R³ and R⁴ in the above formula (2) is not particularly limited, and examples thereof include an alkyl group, an alkenyl group, and an aryl group. Specific examples thereof include those described in the above linking group.

The number of carbon atoms of the hydrocarbon group in R³ and R⁴ is preferably 1 to 4, more preferably 1 to 3, and still more preferably 1 to 2.

The hydrocarbon group in R³ and R⁴ is preferably an alkyl group, more preferably a methyl group, an ethyl group, a n-propyl group, or a n-butyl group, and more preferably a methyl group or an ethyl group.

Specific examples of the compound represented by the formula (2) include: tetrafunctional alkoxysilanes having four alkoxy groups such as tetramethoxysilane, tetraethoxysilane, tetraisopropoxysilane, tetrabutoxysilane, and dimethoxydiethoxysilane; trifunctional alkoxysilane having three alkoxy groups such as methyltrimethoxysilane, methyltriethoxysilane, ethyltrimethoxysilane, and ethyltriethoxysilane; bifunctional alkoxysilane having two alkoxy groups such as dimethyldimethoxysilane, dimethyldiethoxysilane, diethyldimethoxysilane, and diethyldiethoxysilane; and monofunctional alkoxysilane having one alkoxy group such as trimethylmethoxysilane, trimethylethoxysilane, triethylmethoxysilane, and triethylethoxysilane.

These can be used singly or in combination of two or more.

The molecular structure of the silane compound can be specified by NMR (Si, H) measurement after collecting a gel applied on the electrode surface and dissolving the gel with deuterated sodium hydroxide.

The sol-gel matrix preferably includes a curing catalyst in order to promote the hydrolysis and polycondensation reaction of the silane compound.

The curing catalyst is not particularly limited, and examples thereof include a basic catalyst and an acidic catalyst.

Examples of the basic catalyst include amines such as polyethyleneimine, N, N-diethylethanolamine, N, N-dimethylethanolamine, triethanolamine, triethylamine, and 3-morpholinopropylamine.

Examples of the acidic catalyst include hydrogen halides such as hydrochloric acid, nitric acid, sulfuric acid, sulfurous acid, hydrogen sulfide, perchloric acid, hydrogen peroxide, carbonic acid, and carboxylic acids such as formic acid and acetic acid.

The curing catalyst is preferably a basic catalyst, more preferably amines, and still more preferably polyethyleneimine.

The sol-gel matrix preferably further includes a photocurable material. Thereby, the sol-gel matrix can be more sufficiently cured.

The photocurable material is not particularly limited, and examples thereof include: polyfunctional acrylic compounds such as polyethylene glycol diacrylate, polyethylene glycol dimethacrylate, tripropylene glycol triacrylate, bispentaerythritol hexaacrylate, ethylene glycol diacrylate, 1,3-butylene glycol di(meth)acrylate, 1,4-butanediol oligoacrylate, diethylene glycol diacrylate, 1,6-hexanediol oligoacrylate, neopentyl glycol diacrylate, triethylene glycol diacrylate, tripropylene glycol diacrylate, dipropylene glycol diacrylate, cyclohexanedimethanol diacrylate, tricyclodecane dimethanol diacrylate, bisphenol A polyethoxydiacrylate, bisphenol F polyethoxydiacrylate, pentaerythritol tetraacrylate, propoxylated (2) neopentyl glycol diacrylate, trimethylolpropane triacrylate, tris(2-hydroxyethyl) isocyanurate triacrylate, pentaerythritol triacrylate, ethoxylated (3) trimethylolpropane triacrylate, propoxylated (3) glyceryl triacrylate, pentaerythritol tetraacrylate, ditrimethylolpropane tetraacrylate, ethoxylated (4) pentaerythritol tetraacrylate, dipentaerythritol pentaacrylate, 2-(2-ethoxyethoxy)ethyl acrylate, hexadiol diacrylate, 1,6-hexanediol diacrylate, ethoxylated 1,6-hexanediol diacrylate, polypropylene glycol diacrylate, 1,4-butanediol diacrylate, 1,9-nonanediol diacrylate, tetraethylene glycol diacrylate, 2-n-butyl-2-ethyl-1,3-propanediol diacrylate, hydroxypivalic acid neopentyl glycol diacrylate, trimethylolpropane triacrylate hydroxypivalate, ethoxylated phosphoric acid triacrylate, ethoxylated tripropylene glycol diacrylate, neopentyl glycol-modified trimethylolpropane diacrylate, stearic acid-modified pentaerythritol diacrylate, tetramethylolpropane triacrylate, tetramethylolmethane triacrylate, caprolactone-modified trimethylolpropane triacrylate, propoxylate glyceryl triacrylate, tetramethylolmethane tetraacrylate, ethoxylated pentaerythritol tetraacrylate, dipentaerythritol hexaacrylate, caprolactone-modified dipentaerythritol hexaacrylate, dipentaerythritol hydroxypentaacrylate, neopentyl glycol oligoacrylate, trimethylolpropane oligoacrylate, pentaerythritol oligoacrylate, ethoxylated neopentyl glycol di(meth)acrylate, propoxylated neopentyl glycol di(meth)acrylate, tripropylene glycol di(meth)acrylate, ethoxylated trimethylolpropane triacrylate, propoxylated trimethylolpropane triacrylate, and 2-(2-vinyloxyethoxy)ethyl acrylate; and silane coupling agent having a functional group such as an acrylic group, a methacrylic group, a vinyl group, an epoxy group, and a thiol group. These can be used singly or in combination of two or more. Among these, polyfunctional acrylic compounds are preferable, and polyethylene glycol diacrylate and polyethylene glycol dimethacrylate are more preferable.

The content ratio of the photocurable material in the sol-gel matrix is not particularly limited, and is preferably 0.1 mass % to 50 mass % with respect to 100 mass % of the silane compound. The content ratio is more preferably 0.5 mass % to 25 mass %, and still more preferably 1 mass % to 10 mass %.

The sol-gel matrix preferably further includes a photopolymerization initiator. This makes it possible to more efficiently cure the sol-gel matrix.

The photopolymerization initiator is not particularly limited, and examples thereof include acylphosphine oxide-based compounds such as bis(2,4,6-trimethylbenzoyl)phenylphosphine oxide, 2,4,6-trimethylbenzoyldiphenylphosphine oxide, benzoyldiphenylphosphine oxide, benzoyldiethoxyphosphine oxide, 2,4,6-trimethylbenzoyldiethoxyphenylphosphine oxide, and phenylbis(2,4,6-trimethylbenzoyl) phosphine oxide. These can be used singly or in combination of two or more. Among these, an acylphosphine oxide-based compound is preferable, and phenylbis(2,4,6-trimethylbenzoyl) phosphine oxide is more preferable.

The content ratio of the photopolymerization initiator in the sol-gel matrix is not particularly limited, and is preferably 0.1 mass % to 10 mass % with respect to 100 mass % of the photocurable material. The content ratio is more preferably 0.2 mass % to 5 mass %, and still more preferably 0.3 mass % to 3 mass.

### <Electrode Support>

The electrode support included in the enzyme electrode of the present disclosure is a conductive substrate that can be connected to an external circuit and can transmit electrons. The material, shape, and the like of the electrode support are not particularly limited as long as the electrode support has the above properties.

The material of the electrode support may be any conductive material, and examples thereof include carbon materials such as carbon cloth, carbon paper, graphite, glassy carbon, activated carbon, carbon black, and carbon nanotube; metals or alloys such as gold, platinum, copper, palladium, titanium, aluminum, silver, and nickel; and conductive oxides such as SnO₂, ITO, In₂O₃, WO₃, and TiO₂.

For the electrode support, one of these may be composed of a single layer, or may be composed of a laminated structure of two or more layers.

The conductive material is preferably a carbon material.

As the carbon material, glassy carbon, activated carbon, carbon black, and carbon nanotubes are preferable.

In the electrode support, for example, when two or more conductive materials are used, a binder such as a polymer may be used.

The polymer is not particularly limited, and polymers including a fluorine atom, such as polyvinylidene fluoride (PVDF) and polyvinyl fluoride (PVF), copolymers thereof, and copolymers of these monomers and ethylene, styrene, or the like may be used. Further, in addition to polymers such as polystyrene, polyethylene, and polypropylene, examples thereof include hydrophilic polymers such as polyacrylic acid, polylysine, and carboxymethylcellulose, and conductive polymers such as polyaniline, polypyrrole, and polyaniline sulfonic acid as a derivative thereof.

The electrode support may have a flat surface of the substrate or may have irregularities and pores on the surface of the substrate, and preferably has pores.

The size of the pores is not particularly limited, and is preferably 0.1 nm to 100 nm. The size of the pores is more preferably 1 nm to 50 nm.

Hereinafter, an embodiment of an enzyme electrode of the present disclosure will be described with reference to the drawings.

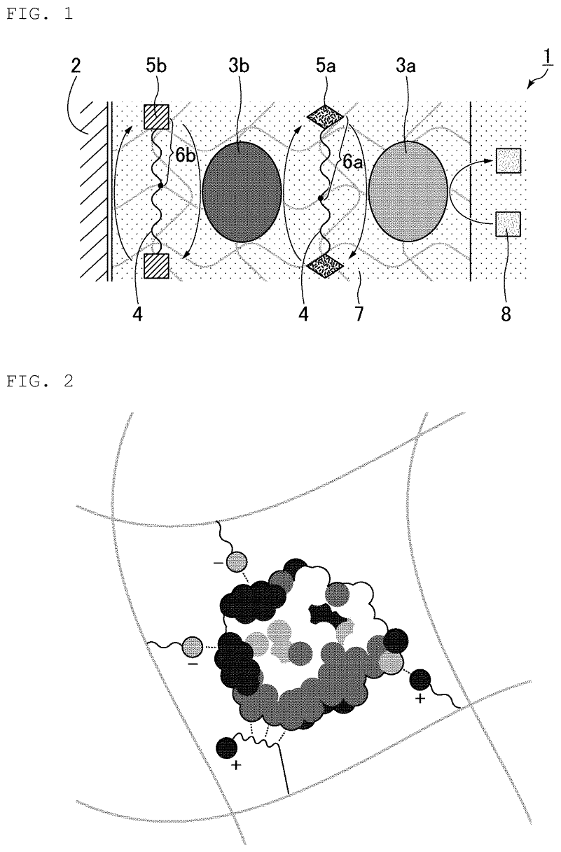

FIG. 1 is a schematic view of an embodiment of an enzyme electrode of the present disclosure. In the enzyme electrode 1, an oxidoreductase (a) 3a, an oxidoreductase (b) 3b, a conjugate 6a of an electron mediator (a) 5a and a silane coupling agent, and a conjugate 6b of an electron mediator (b) 5b and a silane coupling agent are fixed to an electrode support 2.

When the enzyme electrode 1 of the present disclosure is an anode, the electron mediator (a) 5a receives electrons generated by oxidation of the substrate 8 by the oxidoreductase (a) 3a and transfers the received electrons to the oxidoreductase (b) 3b, the oxidoreductase (b) 3b transfers electrons to the electron mediator (b) 5b, the electron mediator (b) 5b transfers electrons to the electrode support 2, and the electrons extracted from the substrate 8 can be transferred to the electrode support 2.

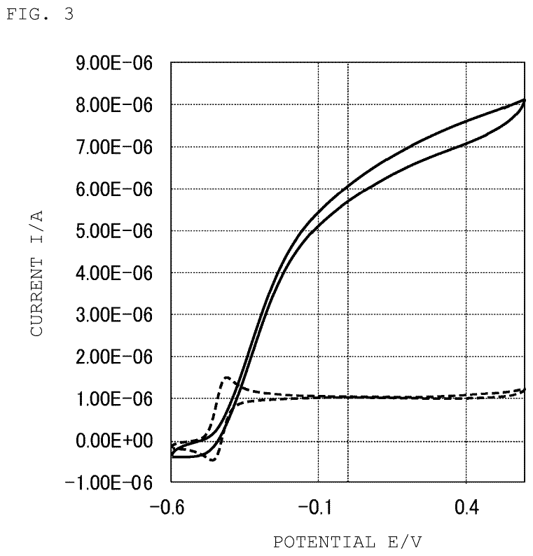

FIG. 2 is a schematic view showing an interaction between a silane coupling agent and an oxidoreductase in an embodiment of the enzyme electrode of the present disclosure.

When the silane coupling agent has a hydrocarbon group having 4 or more carbon atoms in the linking group, the linking group and the hydrophobic moiety of the oxidoreductase form a hydrophobic interaction, allowing the oxidoreductase to be stabilized.

In addition, when the silane coupling agent has a positive or negative charge in the reactive functional group, an electrostatic interaction is formed with a negatively or positively charged portion on the surface of the oxidoreductase, allowing the oxidoreductase to be stabilized.

### [Method for Producing Enzyme Electrode]

The method for producing the enzyme electrode of the present disclosure is not particularly limited, and the enzyme electrode is preferably produced by fixing an oxidoreductase, and a conjugate of a silane coupling agent and an electron mediator to an electrode support with a sol-gel matrix. That is, a method for producing an enzyme electrode, the method including an immobilization step of immobilizing an oxidoreductase, and a conjugate of a silane coupling agent and an electron mediator on an electrode support with a sol-gel matrix is also one of the present disclosure.

Specific examples and preferable forms of the electrode support, the oxidoreductase, the conjugate of a silane coupling agent and an electron mediator, and the sol-gel matrix are as described above.

The immobilization step is not particularly limited as long as the oxidoreductase, the conjugate of the silane coupling agent and the electron mediator are immobilized on the electrode support by the sol-gel matrix, but the immobilization step can be performed by a method in which a composition including the oxidoreductase, the conjugate of the silane coupling agent and the electron mediator, and the sol-gel matrix material is applied onto the electrode support and dried. The application method may be any commonly used method, and examples thereof include a spin coating method, a spray method, a screen method, a dip coating method, and a blade method.

The sol-gel matrix material used in the immobilization step is not particularly limited, but preferably includes a silane compound. Specific examples and preferable forms of the silane compound are as described above.

The composition used in the immobilization step preferably includes a solvent and a buffer component.

Examples of the solvent include aqueous solvents such as water and ethanol. The solvent is preferably water.

Examples of the buffer component include phosphates such as potassium phosphate and sodium phosphate, imidazole, carbonate, borate, tartrate, citrate, tris(hydroxymethyl)aminomethane (TRIS), 4-(2-hydroxyethyl)-piperazine-1-ethanesulfonic acid (HEPES), and 3-morpholinopropanesulfonic acid (MOPS).

The content ratios of the oxidoreductase, the conjugate of the silane coupling agent and the electron mediator, and the sol-gel matrix material in the composition used in the immobilization step can be appropriately adjusted according to these ratios in the enzyme electrode.

The composition used in the immobilization step preferably includes a photocurable material.

In addition, the composition preferably further includes a photopolymerization initiator.

Specific examples and preferable examples of the photocurable material and the photopolymerization initiator are as described above.

In addition, preferable ranges of the amounts of the photocurable material and the photopolymerization initiator used are the same as the content ratio in the sol-gel matrix described above.

When the photocurable material and the photopolymerization initiator are used in the immobilization step, it is preferable to apply the composition onto the electrode support and dry the composition and then perform a light irradiation step.

The light irradiation method can be performed by a commonly used method. The light irradiation conditions vary depending on the energy rays to be used, and for example, in the case of curing by ultraviolet irradiation, the irradiation amount of ultraviolet rays is preferably 10 mJ/cm² to 3,000 mJ/cm², and the irradiation time is preferably 1 second to 180 seconds.

### [Biosensor]

The present disclosure also provides a biosensor including the enzyme electrode of the present disclosure.

The biosensor of the present disclosure is preferably configured to include the enzyme electrode of the present disclosure as a working electrode and a counter electrode thereof.

The measurement by the biosensor is performed by bringing the measurement sample into contact with the biosensor to cause an oxidation-reduction reaction between the oxidoreductase included in the enzyme electrode of the present disclosure and the substance to be measured, and detecting a current generated by the oxidation-reduction reaction. With such a response current value, the presence or absence or concentration of the substrate in the sample can be measured.

Examples of the measurement method using the biosensor of the present disclosure include commonly used methods such as chronoamperometry, coulometry, and cyclic voltammetry for measuring oxidation current or reduction current.

### [Bio Battery]

The present disclosure is also a bio battery including the enzyme electrode of the present disclosure.

The enzyme electrode of the present disclosure in the above bio battery is preferably an anode.

The bio battery of the present disclosure includes the enzyme electrode of the present disclosure, and is not particularly limited as long as the anode and the cathode are connected by an external circuit, and is preferably configured to include a diaphragm that isolates the anode and the cathode.

In one embodiment, the bio battery of the present disclosure is preferably configured to include an anode including the enzyme electrode of the present disclosure, a cathode, and a diaphragm that isolates the anode from the cathode.

As the cathode in the bio battery according to an embodiment of the present disclosure, for example, an enzyme catalyst such as a multicopper enzyme including pyruvate oxidase, ascorbate oxidase, or laccase, or a metal catalyst such as platinum can be used. When the enzyme catalytic mechanism is used for the reaction on the cathode side, the cathode may be preferably configured such that the enzyme is fixed to the electrode support or supplied as an enzyme solution onto an appropriate electrode support without being fixed. In this case, as the electrode support, the electrode support described in the enzyme electrode of the present disclosure can be used in the same manner.

The material, shape, and the like of the diaphragm are not limited as long as the diaphragm has ion conductivity capable of transmitting protons and the like, and has a property of not transmitting constituent components on the negative electrode side and constituent components on the positive electrode side other than ions such as protons. For example, a cellulose membrane or the like can be used, and a solid electrolyte membrane can be used. Examples of the solid electrolyte membrane include, but are not limited to, a solid membrane having an ion exchange function such as an organic polymer having a strong acid group such as a sulfo group, a phosphate group, a phosphon group, and a phosphine group, a weak acid group such as a carboxy group, and a polar group. Specifically, a cellulose membrane and a perfluorocarbon sulfonic acid (PFS)-based resin membrane such as Nafion (registered trademark) that is a copolymer of tetrafluoroethylene and perfluoro [2-(fluorosulfonylethoxy) propyl vinyl ether] can be used.

### [Bioreactor]

The enzyme electrode of the present disclosure can be used as a bioreactor. A bioreactor including an enzyme electrode of the disclosure is also one of the disclosures.

The bioreactor is not particularly limited as long as the enzyme electrode of the present disclosure acts as a reaction site with a reactant, and in one embodiment, the enzyme electrode of the present disclosure is preferably provided in a column reactor.

In one embodiment described above, when the solution including the reactant is flowed through the column reactor and brought into contact with the enzyme electrode, the enzyme reaction by the oxidoreductase included in the enzyme electrode of the present disclosure provides a product from the reactant.

The reactant applied to the bioreactor is not particularly limited as long as it is a substrate oxidized or reduced by an oxidoreductase. Specific examples thereof include the above-described substrate.

### EXAMPLES

Hereinafter, examples for more specifically describing the present disclosure will be described. The present disclosure is not limited only to these examples.

### <Cyclic Voltammetry (CV) Measurement>

CV measurement was performed under the following conditions.

A 1280Z electrochemical measurement system (manufactured by Solartron Analytical) was used for the CV measurement. The solution was placed in an electrochemical measurement cell and kept at 37° C., platinum as a counter electrode, Ag/AgCl as a reference electrode, and a working electrode were immersed in the cell, and a nitrogen gas was passed through the cell for 10 minutes to remove dissolved oxygen in the solution, and then the voltage was continuously changed at a sweep rate of 5 mV/sec between −0.6 V and +0.6 V with respect to Ag/AgCl.

### <Cross-Cut Peeling Test for Measuring Mechanical Strength>

The cross-cut peeling test was performed by the following method using a micro-scratch test.

The composition liquid was dropped onto a glass plate and dried overnight at 4° C., then scratched at 25 μmR and 10 mN, and judgement was performed according to the following criteria.
◯: The base was not exposed after the micro-scratch.
x: The base was exposed.

### Production Example 1: Conjugate of NAD and GOS

25 mg of NAD (manufactured by FUJIFILM Wako Pure Chemical Corporation) and 48.6 mg of 8-glycidoxyoctyltrimethoxysilane (hereinafter also referred to as GOS, manufactured by Shin-Etsu Chemical Co., Ltd.) were mixed in 400 μL of 0.1M Tris-HCl buffer (pH7.5), and the mixture was stirred for 7 hours to obtain a conjugate of NAD and GOS (hereinafter, also referred to as NAD-GOS).

### Production Example 2: Conjugate of ANQ and GOS

25 mg of aminonaphthoquinone (hereinafter, also referred to as ANQ) and 48.6 mg of GOS were mixed in 400 μL of 0.1M Tris-HCl buffer (pH7.5), and the mixture was stirred for 7 hours to obtain a conjugate of ANQ and GOS (hereinafter, also referred to as ANQ-GOS).

### Comparative Production Example 1: Conjugate of NAD and GPS

25 mg of NAD and 37.5 mg of 3-glycidoxypropyltoethoxysilane (hereinafter also referred to as GPS, manufactured by Shin-Etsu Chemical Co., Ltd.) were mixed in 400 μL of 0.1M Tris-HCl buffer (pH7.5), and the mixture was stirred for 7 hours to obtain a conjugate of NAD and GPS (hereinafter, also referred to as NAD-GPS).

### Comparative Production Example 2: Conjugate of ANQ and GPS

25 mg of ANQ and 37.5 mg of GPS were mixed in 400 μL of 0.1M Tris-HCl buffer (pH7.5), and the mixture was stirred for 7 hours to obtain a conjugate of ANQ and GPS (hereinafter, also referred to as ANQ-GPS).

### Production Example 3: Sol-Gel Matrix (I)

0.18 g of tetraethoxysilane (hereinafter also referred to as TEOS, manufactured by Tokyo Chemical Industry Co., Ltd.), 0.5 mL of water, and 0.625 mL of 0.01M hydrochloric acid were mixed and stirred for 7 hours to obtain a sol-gel matrix (I) (hereinafter, also referred to as Sol (I)).

### Production Example 4: Sol-Gel Matrix (II)

0.16 g of TEOS, 0.01 g of methyltriethoxysilane (hereinafter also referred to as MTES, manufactured by Shin-Etsu Chemical Co., Ltd.), 0.01 g of dimethyldiethoxysilane (hereinafter also referred to as DMDES, manufactured by Shin-Etsu Chemical Co., Ltd.), 0.5 mL of water, and 0.625 mL of 0.01M hydrochloric acid were mixed and stirred for 7 hours to obtain a sol-gel matrix (II) (hereinafter, also referred to as Sol (II)).

### Production Example 5: 10% Polyethyleneimine Solution

Polyethyleneimine (hereinafter also referred to as PEI, manufactured by FUJIFILM Wako Pure Chemical Corporation, molecular weight 10,000) and pure water were mixed, and the pH was adjusted to 9 with hydrochloric acid to obtain a 10% polyethyleneimine solution (hereinafter, also referred to as PEIaq).

### Example 1

15 μL of 10 mg/mL glycerol dehydrogenase (hereinafter also referred to as GLDH, manufactured by TOYOBO CO., LTD.), 10 μL of 5 mg/mL diaphorase (hereinafter also referred to as DI, manufactured by FUJIFILM Wako Pure Chemical Corporation), 10 μL of NAD-GOS, 20 μL of Sol (I), and 10 μL of PEIaq were added in this order to an Eppendorf tube and stirred, and then the resulting composition of 10 uL was immediately dropped onto the glassy carbon electrode (hereinafter, also referred to as a GC electrode) and dried overnight at 4° C. to obtain an enzyme electrode 1.

### Comparative Example 1

15 μL of 10 mg/mL GLDH, 10 μL of 5 mg/mL DI, 10 μL of NAD-GPS, 20 μL of Sol (I), and 10 μL of PEIaq were added in this order to an Eppendorf tube and stirred, and then the resulting composition of 10 μL was immediately added dropwise onto the GC electrode and dried overnight at 4° C. to obtain a comparative enzyme electrode 1.

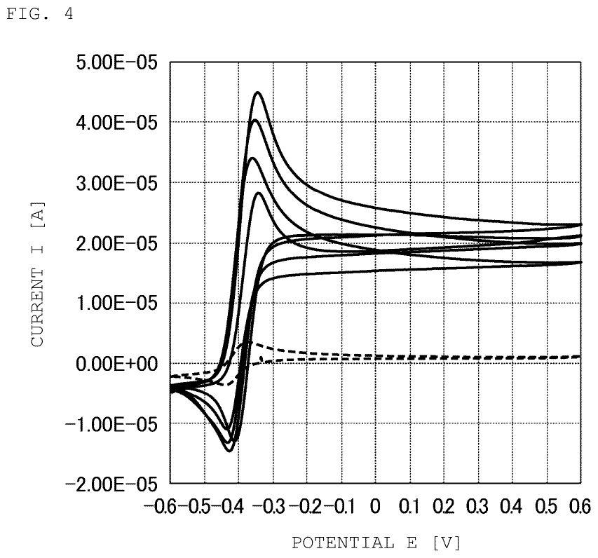

The enzyme electrodes obtained in Example 1 and Comparative Example 1 were subjected to cyclic voltammetry (CV) measurement using an electrochemical measurement cell 1 including 10 ml of 0.1M NH₄OH—NH₄Cl buffer (pH9.0)+500 μL of glycerol+10 mg of ANQ. The measurement results are shown in FIG. 3. In FIG. 3, Example 1 is indicated by a solid line, and Comparative Example 1 is indicated by a broken line.

From the results of FIG. 3, in Example 1, the catalytic current value was improved as compared with Comparative Example 1. In Example 1, it is considered that the silane coupling agent having a linking group having 4 or more carbon atoms was bonded to NAD, thereby improving the mobility of NAD, and increasing the reaction rate between NAD and GLDH or DI.

### Example 2

15 μL of 10 mg/mL GLDH, 10 μL of 5 mg/mL DI, 10 μL of NAD-GOS, 15 μL of ANQ-GOS, 20 μL of Sol (I), and 10 μL of PEIaq were added in this order to an Eppendorf tube, and 10 μL of the composition obtained after stirring was immediately dropped onto the GC electrode and dried overnight at 4° C. to obtain an enzyme electrode 2.

### Example 3

15 μL of 10 mg/mL GLDH, 10 μL of 5 mg/mL DI, 10 μL of NAD-GOS, 15 μL of ANQ-GOS, 20 μL of Sol (II), and 10 μL of PEIaq were added in this order to an Eppendorf tube, and 10 μL of the composition obtained after stirring was immediately dropped onto the GC electrode and dried overnight at 4° C. to obtain an enzyme electrode 3.

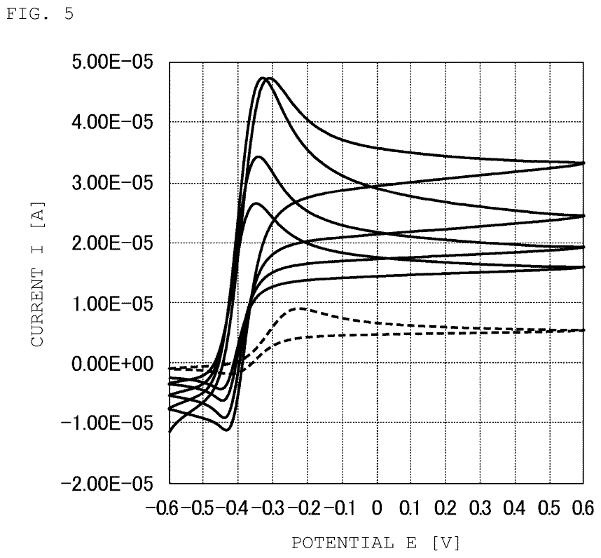

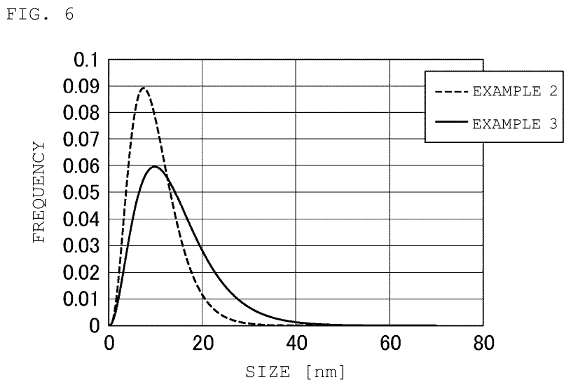

The enzyme electrodes obtained in Examples 2 and 3 were subjected to cyclic voltammetry (CV) measurement using an electrochemical measurement cell 2 including 10 mL of 0.1M NH₄OH—NH₄Cl buffer (pH 9.0)+500 μL of glycerol. The measurement was performed immediately after immersing the enzyme electrode in the electrochemical measurement cell 2 and after 5 hours from the immersion. The measurement results are shown in FIGS. 4 and 5. In FIGS. 4 and 5, the result immediately after immersion is indicated by a solid line, and the result after 5 hours from immersion is indicated by a broken line.

As a result of the measurement, in Examples 2 and 3, the catalytic current values immediately after immersing the enzyme electrode in the electrochemical measurement cell 2 were 2.1×10⁻⁵ A and 3.3×10⁻⁵ A, respectively. In addition, the catalytic current values in Examples 2 and 3 after 5 hours from immersion were 1.2×10⁻⁶ A and 5.4×10⁻⁶ A, respectively. In Example 3, the catalytic current value was improved immediately after immersion and after 5 hours.

The compositions applied onto the electrode surfaces of the enzyme electrodes obtained in Examples 2 and 3 were analyzed by small-angle X-ray scattering (SAXS) using NANOPIX manufactured by Rigaku Corporation. The measurement results are shown in FIG. 6. In FIG. 6, Example 2 is indicated by a broken line, and Example 3 is indicated by a solid line. As a result of the measurement, the average size of the pores of the gel of Example 2 in which only the tetrafunctional alkoxysilane was used was 10 nm (a network structure with a 10 nm periodicity was formed), whereas the average size of the pores of the gel of Example 3 in which the bifunctional alkoxysilane and the trifunctional alkoxysilane were further added was 14 nm (a network structure with a 14 nm periodicity was formed), and it could be confirmed that the molecular network in the sol-gel matrix was expanded in Example 3.

### Example 4

0.1 g of polyethylene glycol dimethacrylate (hereinafter also referred to as PEGDMA, manufactured by FUJIFILM Wako Pure Chemical Corporation) and 1 mg of phenylbis(2,3,4-trimethylbenzoyl) phosphine oxide (manufactured by IGM RESINS) were added to Sol (I) prepared in Production Example 3 to obtain Sol (I′). 10 μL of the composition prepared in the same manner as in Example except that the obtained Sol (I′) was used in place of Sol (I) was dropped onto a glass substrate, dried, and then irradiated with ultraviolet rays. The obtained glass plate was subjected to a cross-cut peeling test, and the mechanical strength of the matrix was evaluated according to the evaluation criteria described above. The evaluation result was ◯.

Using the photocurable material in the sol-gel matrix improves the mechanical strength of the matrix, and the sol-gel matrix was hardly peeled off from the electrode surface. This is considered to improve the durability of the electrode.

### DESCRIPTION OF REFERENCE SYMBOLS

1: Enzyme electrode
2: Electrode support
3a: Oxidoreductase (a)
3b: Oxidoreductase (b)
4: Silane coupling agent
5a: Electron mediator (a)
5b: Electron mediator (b)
6a: Conjugate of silane coupling agent and electron mediator (a)
6b: Conjugate of silane coupling agent and electron mediator (b)
7: Sol-gel matrix
8: Substrate

1. An enzyme electrode comprising:
an electrode support;
an oxidoreductase;
a conjugate of a silane coupling agent and an electron mediator; and
a sol-gel matrix, wherein
the oxidoreductase and the conjugate are fixed to the electrode support by the sol-gel matrix, and
the silane coupling agent has a silicon atom, a reactive functional group, and a hydrolyzable group, and a structure in which the silicon atom and the reactive functional group are linked by a linking group having 4 or more carbon atoms.

2. The enzyme electrode according to claim 1, wherein the oxidoreductase comprises NAD(P)H or an NAD(P)-dependent oxidase and diaphorase.

3. The enzyme electrode according to claim 2, wherein the electron mediator comprises NAD(P)H and/or NAD(P).

4. The enzyme electrode according to claim 1, wherein the electron mediator comprises NAD(P)H and/or NAD(P).

5. The enzyme electrode according to claim 1, wherein the silane coupling agent has a positive charge or a negative charge.

6. The enzyme electrode according to claim 1, wherein the sol-gel matrix includes a silane compound.

7. The enzyme electrode according to claim 1, wherein a content ratio of the conjugate of the silane coupling agent and the electron mediator in the enzyme electrode is 1,000 mol % to 10 million mol % with respect to 100 mol % of the oxidoreductase.

8. The enzyme electrode according to claim 1, wherein the reactive functional group in the silane coupling agent and a reactive functional group in the electron mediator are bonded by a covalent bond.

9. The enzyme electrode according to claim 1, wherein the linking group is a hydrocarbon group having 4 to 30 carbon atoms.

10. The enzyme electrode according to claim 1, wherein the electrode support is a conductive substrate.

11. A method for producing an enzyme electrode, the method comprising:
immobilizing an oxidoreductase, and a conjugate of a silane coupling agent and an electron mediator on an electrode support by a sol-gel matrix,
wherein the silane coupling agent has a silicon atom, a reactive functional group, and a hydrolyzable group, and has a structure in which the silicon atom and the reactive functional group are linked by a linking group having 4 or more carbon atoms.

12. The method for producing an enzyme electrode according to claim 11, wherein during the immobilization, the sol-gel matrix is cured using a photocurable material.

13. The method for producing an enzyme electrode according to claim 11, wherein the oxidoreductase comprises NAD(P)H or an NAD(P)-dependent oxidase and diaphorase.

14. The method for producing an enzyme electrode according to claim 13, wherein the electron mediator comprises NAD(P)H and/or NAD(P).

15. The method for producing an enzyme electrode according to claim 11, wherein the electron mediator comprises NAD(P)H and/or NAD(P).

16. The method for producing an enzyme electrode according to claim 11, wherein the silane coupling agent has a positive charge or a negative charge.

17. The method for producing an enzyme electrode according to claim 11, wherein the sol-gel matrix includes a silane compound.

18. A biosensor comprising the enzyme electrode according to claim 1.

19. A bio battery comprising the enzyme electrode according to claim 1.

20. A bioreactor comprising the enzyme electrode according to claim 1.
